(19) United States
(12) Patent Application Publication
(10) Pub. No.: US 2020/0000541 A1
(43) Pub. Date: Jan. 2, 2020

(54) DENTAL SPLATTER SHIELD WITH FLUIUD ABSORPTION AND LIGHT REFLECTION

(71) Applicant: Gail Kathleen Clemens, Henderson, NV (US)

(72) Inventor: Gail Kathleen Clemens, Henderson, NV (US)

(21) Appl. No.: 16/456,026

(22) Filed: Jun. 28, 2019

Related U.S. Application Data
(60) Provisional application No. 62/763,812, filed on Jul. 2, 2018.

(51) Int. Cl. A61B 90/00; A61C 17/02; A61C 17/06

(52) U.S. Cl. CPC A61B 90/05; A61B 2017/0023; A61C 17/06; A61C 17/0208

(57) ABSTRACT

A dental instrument is described. The dental instrument includes a shaft including a graspable end and a usable end. The dental instrument also includes a splatter shield. The splatter shield comprises a pliable sheet material having within the sheet material an aperture formed therethrough. The aperture is sized and shaped in a way to be frictionally and selectively coupled to the usable end of the shaft of the dental instrument.

### REFERENCE TO RELATED APPLICATIONS

This application claims priority to U.S. Provisional Application No. 62/763,812 filed on Jul. 2, 2018 and entitled DENTAL SPLATTER SHIELD WITH FLUID ABSORPTION AND LIGHT REFLECTION to inventor Gail Kathleen Clemens which is herein incorporated by reference in its entirety.

### BACKGROUND

During conventional dental procedures dental hygienists, dental assistants, dentists, orthodontists, and other people working directly with patients' open mouths run the risk of being exposed to infection through airborne bacteria dispersed in the mouth during different dental, medical, or other open mouth procedures. According to the Center for Disease Control, airborne bacteria is a pathway for the transmission of Severe Acquired Respiratory Syndrome (SARS), Tuberculosis (TB), measles, mumps, influenza, human immunodeficiency virus (HIV), other viruses and bacteria, etc. The Center for Disease Control and the American Dental Association consensus has been that the greatest airborne infection threat in dentistry comes from the aerosols due to their ability to stay airborne and potential to enter respiratory passages. Any dental procedure with potential to aerosolize saliva and blood will spread infection bacteria from the mouth, nose, throat, nasopharynx and respiratory tract. When dental procedures are being carried out, the use of some dental tools often cause aerosolization of saliva putting any people near the mouth at risk.

Accordingly, there is a need for devices and apparatuses which aid in preventing splatter of liquids and aerosols of liquid from the mouth and coming into contact with the dental workers.

### SUMMARY

An exemplary embodiment relates to a dental instrument. The dental instrument includes a shaft including a graspable end and a usable end. The dental instrument also includes a splatter shield. The splatter shield comprises a pliable sheet material having within the sheet material an aperture formed therethrough. The aperture is sized and shaped in a way to be frictionally and selectively coupled to the usable end of the shaft of the dental instrument.

Another exemplary embodiment relates to a splatter shield for a dental instrument. The splatter shield includes a sheet material. The splatter shield also includes an aperture formed through the sheet material. The aperture is sized and shaped in a way to be frictionally and selectively coupled to the usable end of the shaft of a dental instrument. The size and shape of the sheet material is such that it will deflect some splatter from a dental patient's mouth when coupled to and used with the dental instrument and sized and shaped to minimize interference for the dental instrument user.

Yet another exemplary embodiment relates to a method of limiting the spread of bacteria during a dental procedure. The method includes providing a dental instrument to be used with a dental patient. The method also includes providing a splatter shield for a dental instrument. The splatter shield comprises a sheet material. An aperture is formed through the sheet material. The aperture is sized and shaped in a way to be frictionally and selectively coupled to the usable end of the shaft of a dental instrument, and the size and shape of the sheet material is such that it will deflect some splatter from a dental patient's mouth when coupled to and used with the dental instrument and sized and shaped to minimize interference for the dental instrument user. Further, the method includes placing the splatter shield onto the shaft of the dental instrument by inserting the shaft through the aperture. Further still, the method includes using the dental instrument on the dental patient. Yet further still, the method includes removing the splatter shield from the shaft of the dental instrument and disposing of the splatter shield.

In addition to the foregoing, other system aspects are described in the claims, drawings, and text forming a part of the disclosure set forth herein. The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the disclosures set forth herein.

### BRIEF DESCRIPTION OF THE DRAWINGS

FIG. 1 is an exemplary embodiment of dental instrument, including a splatter shield.

FIG. 2 is an exemplary embodiment of a dental instrument including a splatter shield being used with a dental patient.

FIG. 3 is an exemplary embodiment of a particular configuration of a splatter shield:

FIG. 4 is an exemplary embodiment of an alternative configuration of a splatter shield.

The use of the same symbols in different drawings typically indicates similar or identical items unless context dictates otherwise.

### DETAILED DESCRIPTION

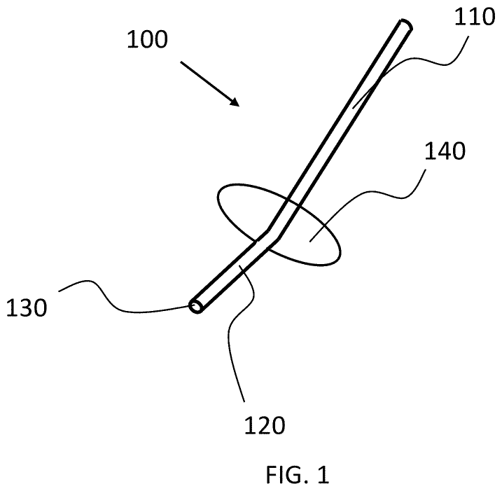

Referring to FIG. 1, a dental tool 100 such as but not limited to a saliva suctioning device, a toothbrush, a drill, a water pick, an irrigator, etc., includes a handle portion 110, a tip portion 120 and a tool portion, exit portion, or entrance portion 130. In accordance with an exemplary embodiment, dental tool 100 is fitted with a disposable splatter shield 140 that has been slid onto tip portion 120. Splatter shield 140 is a solution to reduce the amount of bacteria in the mouth going airborne during various dental procedures and transmitting bacteria airborne in the mouth and outside of the mouth. Studies show that many different dental procedures emit airborne bacteria and blood creating a cloud. This not only can affect the patient but also the clinician. It has been observed that airborne bacteria can travel as far as 18 inches outside of the mouth.

Splatter shield 140 acts as a barrier attached to dental instruments to reduce or detract from the amount of airborne bacteria. In accordance with exemplary embodiments, splatter shield 140 may be formed in different sizes, shapes, and materials. Splatter shield 140 may be formed with a hole or slits to accommodate insertion into the middle of splatter shield 140 to slide onto tip portion 120 of dental instrument 100 creating an umbrella like barrier. In accordance with an exemplary embodiment the material forming splatter shield 140 must be resilient enough to be comfortable when abutting to mouth soft tissue. The entry insertion of the barrier may be different sizes and shapes adaptable for easy entry of any size or shape of dental instruments. In accordance with an exemplary embodiment splatter shield 140 is preferably disposable but it may also be a material that is able to be sterilized and thus reusable.

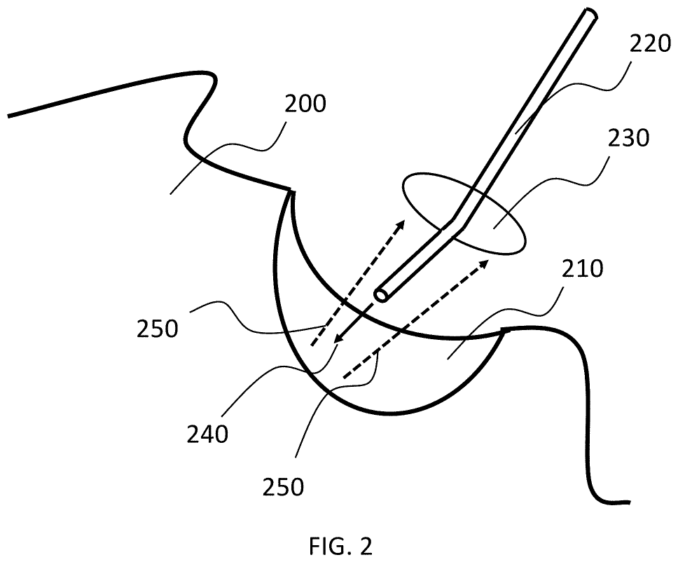

Referring now to FIG. 2, a dental patient 200 is depicted with an open mouth cavity 210. A dental instrument 220, such as an air water syringe 220 is depicted with a splatter shield 230 positioned above mouth cavity 210. Air water syringe 220 ejects water and air in a direction 240 in to mouth cavity 210. Often water and bacteria may be reflected out of mouth cavity 210 in directions 250. In this instance splatter shield 230 may either absorb or reflect some of the water and bacteria moving in directions 250. The reflection or absorption by splatter shield 230 may help to protect dental workers from coming in contact with some of the bacteria.

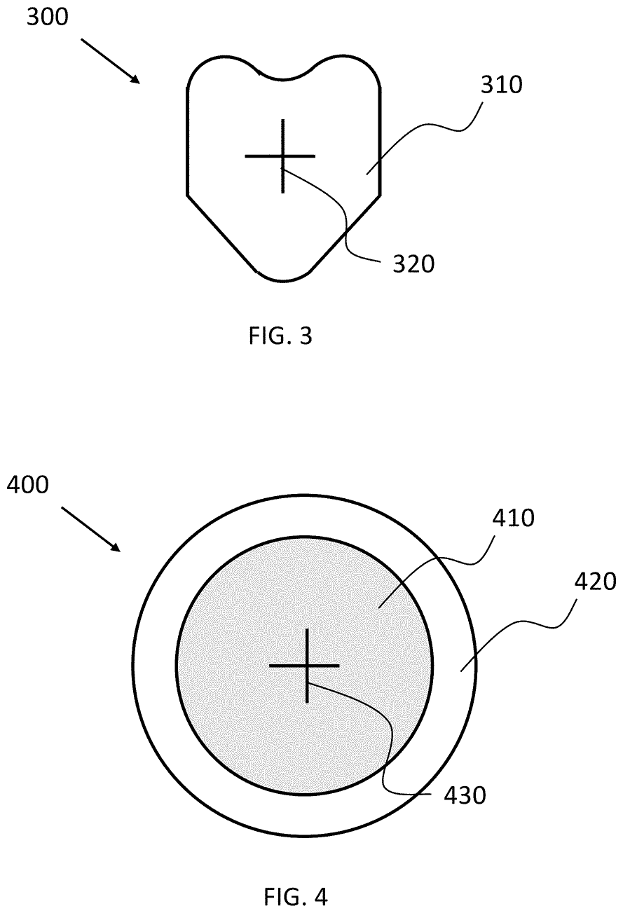

In accordance with a preferred exemplary embodiment, splatter shield 230 may be a fluid absorbing material or alternatively a thin plastic material that can transmit light therethrough while deflecting and reducing the amount of airborne bacteria and blood or any combination of materials. Splatter shield 230 may be added on to the instrument for single-use but may alternatively be manufactured into the instrument. Referring now to FIG. 3, an example of a splatter shield 300 is depicted. Splatter shield 300 is of a particular shield shape, but is not limited thereto. Splatter shield 300 may be of any of a variety of shapes including but not limited to a circle, a square, a rectangle, a pentagon, a hexagon, or any other shape. Splatter shield 300 is depicted as a thin layer of material 310 such as clear or translucent plastic. Splatter shield 300 includes a receiving aperture 320. Receiving aperture 320 is depicted as two slits in material layer 310 configured in a crossed pattern. Receiving aperture 320 may also be a hole through the material of any sort that is configured to frictionally grip onto and accommodate the shaft of a dental instrument or tool that is inserted through the aperture.

Referring now to FIG. 4, an alternative embodiment of a splatter shield 400 is depicted. Splatter shield 400 is configured as a combined material circular splatter shield. In accordance with a particular embodiment, splatter shield 400 may be configured as a combined material splatter shield with a first absorbent material 410 and a second clear plastic material 420. Clear plastic material 420 may be both see-through as well as soft and flexible so that it does not bother the patient if it is pressed against the patient's mouth. Splatter shield 400 also includes a receiving aperture 430 depicted here as two crossed slits. In some particular embodiments the edges 420 of splatter shield 400 may be raised to create a cup-like splatter shield. Many other shapes, sizes and configurations of the splatter shields may be used without departing from the scope of the invention.

Referring again to FIG. 2, the type of dental instrument or tool 220 that is applicable to use with splatter shield 230 are ones that are used in the mouth and come in contact or create a water aerosol, these instruments are but are not limited to a saliva ejector, a mouth mirror, an air water syringe tip, a cavitron tip, a prophy angle, a high volume evacuator (HVE), etc. Splatter shield 230 will not completely eliminate airborne bacteria dispersal but will help reduce the amount and risk to dental personnel. Using splatter shield 220 on many applicable instruments during a patient visit will exponentially reduce the amount of airborne bacteria.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

What is claimed is:

1. A dental instrument comprising:
a shaft including a graspable end and a usable end;
a splatter shield, the splatter shield comprising a pliable sheet, material having within the sheet material an aperture formed therethrough, the aperture sized and shaped in a way to be frictionally and selectively coupled to the usable end of the shaft of the dental instrument.

2. The dental instrument of claim 1, wherein the shaft is at least one of a saliva ejector, a mouth mirror, an air water syringe tip, a cavitron tip, a prophy angle, and a high volume evacuator (HVE).

3. The dental instrument of claim 1, wherein the splatter shield is formed of substantially clear plastic.

4. The dental instrument of claim 1, wherein the splatter shield is formed of ore than one material.

5. The dental instrument of claim 1, wherein the splatter shield is formed of more than one material and one of the more than one material is an absorbent material.

6. The dental instrument of claim 1, wherein the splatter shield is formed of more than one material where one of the more than one material is an absorbent material and another of the more than one material is a clear plastic.

7. The dental instrument of claim 1, wherein the aperture comprises one or more slits in the sheet material.

8. The dental instrument of claim 1, wherein the aperture comprises a substantially circular hole in the sheet material.

9. The dental instrument of claim 1, wherein the splatter shield is configured to be disposable.

10. The dental instrument claim 1, wherein he splatter shield is configured to be sterilizable.

11. A splatter shield for a dental instrument, comprising:
a sheet material;
an aperture formed through the sheet material, the aperture sized and shaped in a way to be frictionally and selectively coupled to the usable end of the shaft of a dental instrument,
wherein the size and shape of the sheet material is such that it will deflect sore splatter from a dental patient's mouth when coupled to and used with the dental instrument and sized and shaped to minimize interference for the dental instrument user.

12. The splatter shield for a dental instrument of claim 11, wherein the splatter shield is configured to couple with at least one of a saliva ejector, a mouth mirror, an air water syringe tip, a cavitron tip, a prophy angle, and a high volume evacuator (HVE).

13. The splatter shield for a dental instrument of claim 11, wherein the splatter shield is formed of substantially clear plastic.

14. The splatter shield for a dental instrument of claim 11, wherein t e splatter formed of more than one material.

15. The splatter shield for a dental instrument of claim 11, wherein the splatter shield is formed of more than one material and one of the more than one material is an absorbent material.

16. The splatter shield for a dental instrument of claim 11, wherein the splatter shield is formed of more than one material where one of the more than one material is an absorbent material and another of the more than one material is a clear plastic.

17. The splatter shield for a dental instrument of claim 11, wherein the aperture comprises one or more slits in the sheet material.

18. The splatter shield for a dental instrument of claim 11, wherein the aperture comprises a substantially circular hole in the sheet material.

19. The splatter shield for a dental instrument of claim 11, wherein the splatter configured to be disposable.

20. A method of limiting the spread of bacteria during a dental procedure, comprising:
providing a dental instrument to be used with a dental patient;
providing a splatter shield for a dental instrument the splatter shield comprising a sheet material, an aperture formed through the sheet, material, the aperture sized and shaped in a way to be frictionally and selectively coupled to the usable end of the shaft of a dental instrument, and the size and shape of the sheet material is such that it will deflect some splatter from a dental patient's mouth when coupled to and used with the dental instrument and sized and shaped to minimize interference for the dental instrument user;
placing the splatter shield onto the shaft of the dental instrument by inserting the shaft through the aperture;
using the dental instrument on the dental patient; and
removing the splatter shield from the shaft of the dental instrument; and
disposing of the splatter shield.
